(19) United States
(12) Patent Application Publication
(10) Pub. No.: US 2005/0042185 A1
(43) Pub. Date: Feb. 24, 2005

(54) Whitening compound

(76) Inventor: Jingwei Zhao, Anaheim, CA (US)

Correspondence Address: Raymond Y. Chan;Suite 128, 108 N. Ynez Ave., Monterey Park, CA, 91754, US

(21) Appl. No.: 10/644,612

(22) Filed: Aug. 19, 2003

(51) Int. Cl. A61K 7/20; A61K 7/26

(52) U.S. Cl. 424/53; 424/58

(57) ABSTRACT

A tooth whitening compound includes a predetermined amount of whitening agent and whitening catalyst, wherein the whitening compound includes, a predetermined composition by weight water of 50% to 70%; poloxamer 407 of 10% to 30%; hydrogen peroxide of 3% to 6%; glycerin of 7% to 10%; dicalcium phosphate dihydrate of 1% to 2%; a predetermined kind of flavor of 1%; aloe vera of 0.1% to 1%; phosphoric acid of 1% to 2%; Food, Drug & Cosmetic Green No. 3 of 0.1% to 1%; and mulberry root extract of 0.1% to 1%. The whitening catalyst is the mulberry root extract as a teeth whitening catalyst for enhancing effectiveness, efficiency and stability of teeth whitening process of the whitening compound.

### BACKGROUND OF THE PRESENT INVENTION

1. Field of Invention

The present invention relates to tooth whitening gel, and more particularly to a kind of tooth whitening gel which comprises mulberry root extract to substantially enhance the function of the tooth whitening gel.

2. Description of Related Arts

Various forms of whitening, such as tooth whitening and skin whitening, have become very popular among youngsters all over the world. The underlying reasons for its popularity may be that teeth whitening enhance appearance, and image of pleasant and healthy character.

Conventionally, options for whitening teeth include toothpaste, mouth rinses, chewing gums, in-office bleaching, tooth whitening solutions, and the likes. Despite the existence of various means for teeth whitening, the respective principle is pretty much the same. A tooth whitening substance is typically applied on or mixed with a predetermined carrying agent, such as a certain amount of regular toothpaste, so that when the carrying agent is placed into contact with the surface of one particular teeth, the tooth whitening substance will be in chemically communication with teeth's surface, usually for a prolonged period of time, or may be of a very short period of time in case of whitening toothpaste, so as to provide whitening reaction to the teeth surface.

One skilled in the art would easily appreciate that conventional tooth whitening substance, such as the most widely used peroxides (hydrogen peroxide or Carbamide peroxide), are notorious for the purpose of teeth whitening for its high reactivity. Moreover, it is the high reactivity of such peroxides as a teeth whitening substance which makes conventional teeth whitening process a rather slow or time-consuming process.

Leading researchers have found that the main reasons for its instability hinges on the surface area to volume ratio. This is because the tooth whitening substance is typically stored as a thin layer and not in a syringe or in bulk, as such, for example, it may be that heterogeneous decomposition of hydrogen peroxide becomes a major contributor to the overall decomposition rate of the peroxide. One skilled in the art would also appreciate that it has been reported that the decomposition rates of hydrogen peroxide solutions is roughly proportional to the surface area to volume ratio over a considerable range. As the surface area to volume ratio increases, the decomposition rate increases as well.

Given the conventional difficulty of teeth whitening substance, the present invention provides a teeth whitening gel which enhances the conventional whitening process and effectiveness.

### SUMMARY OF THE PRESENT INVENTION

A main object of the present invention is to provide a gel form teeth whitening compound which comprises a predetermined amount of mulberry root extract as a teeth whitening catalyst for enhancing effectiveness, efficiency and stability of teeth whitening process of the teeth whitening gel.

Another object of the present invention is to provide a teeth whitening compound, wherein the mulberry root extract is specifically used in the teeth whitening gel as a key ingredient for stabilizing the teeth whitening process of the teeth whitening gel.

Another object of the present invention is to provide a process of manufacturing a kind of teeth whitening gel which comprises a predetermined amount of mulberry root extract as a teeth whitening catalyst for enhancing effectiveness, efficiency, and stability of the teeth whiting process.

Another object of the present invention is to provide a process of preparing mulberry root exact for using in the teeth whitening gel to provide effective, efficient and stable teeth whitening process by the teeth whitening gel.

Accordingly, in order to accomplish the above objects, the present invention provides a tooth whitening compound for whitening a tooth surface, comprising:
a whitening agent comprising an active peroxide for applying on said tooth surface to process a teeth whitening reaction between the whitening agent and the tooth surface, and a whitening catalyst having a predetermined amount of amino acids mixing with the whitening agent to chemically react with the active peroxide for stabilizing the teeth whitening reaction.

The whitening agent comprises a poloxamer, a dicalcium phosphate dehydrate, a phosphoric acid, and Food, Drug & Cosmetic (FD&C) Green No.3 mixing together with a predetermined mount of water to form a whitening gel.

Alternatively, the whitening agent comprises an ultrapeg, a glycerin natural kosher, a dicalcium phosphate dihydrate, a carbopol, a phosphoric acid, and a sucralose mixing together with a predetermined mount of water to form a whitening gel.

These and other objectives, features, and advantages of the present invention will become apparent from the following detailed description, the accompanying drawings, and the appended claims.

### BRIEF DESCRIPTION OF THE DRAWINGS

FIG. 1 is a schematic diagram of a predetermined kind of whitening compound according to a first preferred embodiment of the present invention, illustrating the key ingredients of the whitening compound.

FIG. 2 is a flow diagram of a method of preparing the mulberry root extract according to the above first preferred embodiment of the present invention.

FIG. 3 is a flow diagram of a method of manufacturing the whitening compound according the above first preferred embodiment of the present invention.

FIG. 4 is a schematic diagram of a predetermined kind of whitening compound according to an alternative mode of the above first preferred embodiment of the present invention, illustrating the key ingredients of the whitening compound.

FIG. 5 is a flow diagram of a method of manufacturing the whitening compound according the above second preferred embodiment of the present invention.

### DETAILED DESCRIPTION OF THE PREFERRED EMBODIMENT

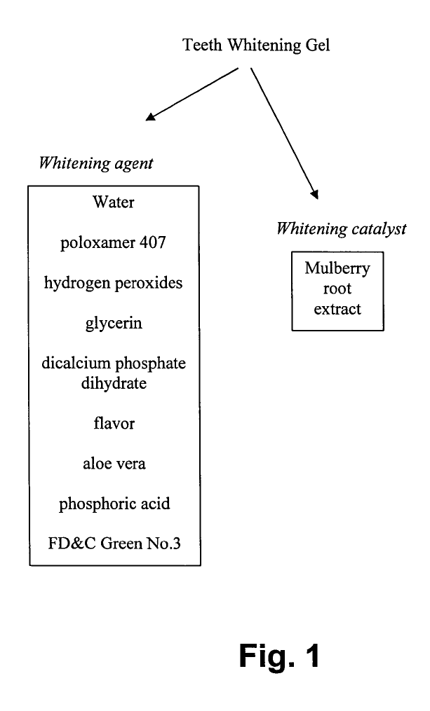

Referring to FIG. 1 of the drawings, the key ingredients of a predetermined kind of whitening compound is shown. According to the first preferred embodiment, the whitening compound for whitening a tooth surface, which is embodied as a kind of teeth whitening gel, comprises a predetermined amount of whitening agent which is mainly responsible for teeth whitening process, and a predetermined amount of whitening catalyst which is mainly responsible for stabilizing and enhance the quality and efficiency of the teeth whitening process.

The whitening agent comprises a predetermined amount of water, poloxamer 407, an active peroxide, such as hydrogen peroxide, glycerin, dicalcium phosphate dihydrate, a predetermined kind of artificial flavor, aloe vera, phosphoric acid, and Food, Drug & Cosmetic (FD&C) Green No.3. The whitening process is preferably triggered by an active reaction between the hydrogen peroxide and a surface of a particular tooth, the rate of reaction being governed by various whitening parameters, such as the ratio of tooth surface area to the volume of the whitening compound. On the other hand, the whitening catalyst comprises a predetermined amount of mulberry root extract which is utilized to stabilize the active reaction involved in the teeth whitening process.

The respective composition by weight of the ingredients of the whitening compound is tabulated in table 1 below:

TABLE 1

| | Composition by |
|---|---|
| Ingredients | weight (% range) |
| water | 50% to 70% |
| poloxamer 407 | 10% to 30% |
| hydrogen peroxide | 3% to 6% |
| glycerin | 7% to 10% |
| dicalcium phosphate dihydrate | 1% to 2% |
| artificial flavor | 1% |
| Aloe vera | 0.1% to 1% |
| phosphoric acid | 1% to 2% |
| Food, Drug and Cosmetic (FD&C) Green | 0.1% to 1% |
| No. 3 | |
| mulberry root extract | 0.1% to 1% |

The whitening compound is preferably in a physical state of a jelly-like substance having a predetermined viscosity, and is adapted to be applied on a surface of a particular teeth or a plurality of tooth in the form of a thin layer, wherein the whitening process could take place on the surfaces of the tooth, the enamel and/or the dentin. The amount of whitening compound to be applied would inevitably depend on the concentration of the exact concentration of the compound and the desirable whitening effect which is to be achieved. Preferably, the teeth whitening gel is to be uniformly, homogeneously, and continuously coated onto the respective teeth for whitening purpose. According to the first preferred embodiment, the teeth whitening gel, in the form of a jelly-like substance, will provide adhesive property so that no external gel carrying member, such as a whitening strip, is required. However, a whitening strip may nevertheless be utilized for the purpose of avoiding the whitening process from being interfered by external objects, such as the internal surface of two lips.

The whitening catalyst, which is embodied as a predetermined amount of mulberry root extract, functions as a catalyst for teeth whitening process. According to the first preferred embodiment, the mulberry root extract comprises a predetermined amount of amino acids which is adapted to react with the hydrogen peroxides of the whitening agent so as to extend and stabilize the teeth whitening reaction between the whitening agent and the respective teeth when the former is applied in such a manner as described above on the later. It is worth to mention that the peroxide in the conventional tooth whitening agent is unstable that the teeth whitening reaction thereof is ineffective. When the whitening catalyst is reacted with whitening compound, the interaction between the peroxide of the whitening agent and the amino acid of the whitening catalyst is adapted to efficiently extend the teeth whitening reaction on the tooth surface in a stable manner.

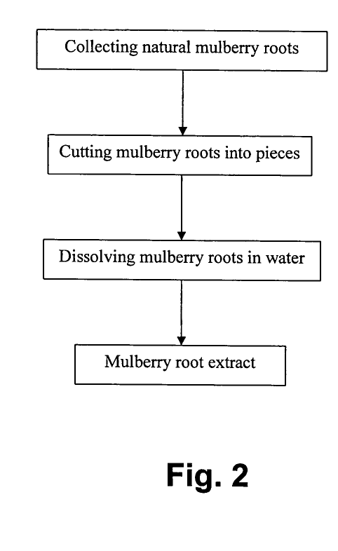

Referring to FIG. 2 of the drawings, a method of preparing the whitening catalyst, specifically embodied as the mulberry roots extracts, according to the first preferred embodiment of the present invention is illustrated. According to the first preferred embodiment, the method of preparing the mulberry roots extracts comprises the steps of:
(a) providing a predetermined amount of natural mulberry roots;
(b) dissolving the natural mulberry roots in water for a predetermined period of time to form a mulberry roots extract solution; and
(c) producing a predetermined amount of mulberry roots extracts from the mulberry roots extract solution.
The step (a) further comprising the sub-steps of:
(a.1) collecting a predetermined amount of natural mulberry roots; and
(a.2) cutting the natural mulberry roots into pieces of predetermined size by a cutting tool.
For the above preparing method, step (c) comprises the steps of:
(c.1) filtering the mulberry roots from the mulberry roots extract solution;
(c.2) evaporating water from said mulberry roots extract solution to form saturated mulberry roots solution; and
(c.3) crystallizing the saturated mulberry roots solution to form the mulberry roots extract.

In step (a) above, the cutting tool is preferably a blender or a manual cutter so that a predetermined amount of mulberry roots pieces are cut in desirable size. Of course, any other cutting tools which are capable of cutting mulberry roots into the above-mentioned form may also be used.

In step (c) above, the predetermined period of time is preferably three days, according to the first preferred embodiment of the present invention.

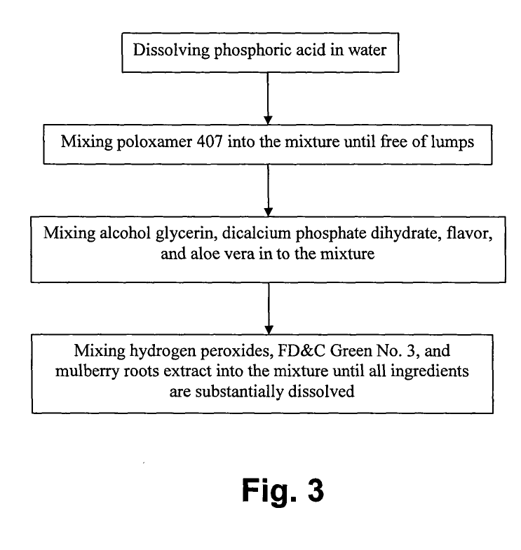

Referring to FIG. 3 of the drawings, a method of manufacturing the whitening compound according to the first preferred embodiment of the present invention is illustrated. The manufacturing method comprises the steps of:
(1) Mix a predetermined amount of the phosphoric acid of predetermined concentration with a predetermined amount of water to form a phosphoric acid solution.
(2) Mix a predetermined amount of the poloxamer 407 with the phosphoric acid solution to form a mixture with free of lumps.
(3) Mix a predetermined respective amount of alcohol, glycerin, dicalcium phosphate dihydrate, artificial flavor, and aloe vera, hydrogen peroxides, and FD&C Green No. 3, in to the mixture until all ingredients are substantially dissolved to form the whitening agent.
(4) Mix the mulberry roots extract with the whitening agent to form the tooth whitening compound.

In the above manufacturing method, in step (3), the artificial flavor and aloe vera in gel form are preferred to mix with the whitening agent to enhance the whitening compound in practice use. Thus, the artificial flavor is preferably a predetermined amount of peppermint so that in addition to teeth whitening, the teeth whitening gel of the present invention could as well deliver a fresh and cool feeling to a user of the present invention.

In step (4), the eventual mixture forms the teeth whitening gel of the present invention, wherein the teeth whitening gel, as mentioned above, is adapted to be applied in a predetermined amount onto the teeth/tooth of the user.

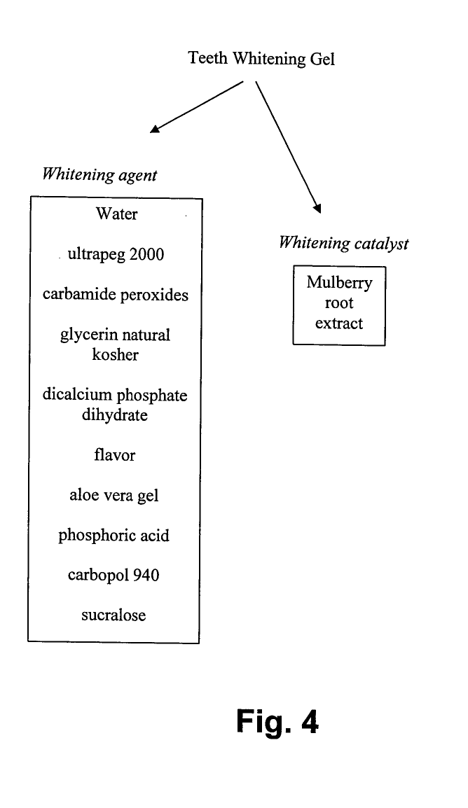

Referring to FIG. 4 of the drawings, an alternative mode of the whitening compound according to the above first preferred embodiment of the present invention is illustrated. According to the first alternative mode, the whitening agent comprises a predetermined amount of water, carbamide peroxide, ultrapeg 2000, glycerin natural kosher, carbopol 940, dicalcium phosphate dihydrate, a predetermined kind of artificial flavor, aloe vera gel, phosphoric acid, and sucralose. The whitening process is preferably triggered by an active reaction between the carbamide peroxide and a surface of a particular tooth, the rate of reaction being governed by various whitening parameters, such as the ratio of tooth surface area to the volume of the whitening compound. On the other hand, as in the case of the first preferred embodiment, the whitening catalyst comprises a predetermined amount of mulberry root extract which is utilized to stabilize the active reaction involved in the teeth whitening process. Preferably, the mulberry root extract is prepared by the method stated above step (a) through step (c).

The respective composition by weight of the ingredients of the whitening compound according to the first alternative mode is tabulated in table 2 below:

TABLE 2

| | Composition by |
|---|---|
| Ingredients | weight (% range) |
| water | 20% to 50% |
| carbamide peroxide | 8% to 22% |
| Ultrapeg 2000 | 20% to 30% |
| glycerin natural kosher | 10% to 20% |
| carbopol 940 | 10% to 30% |
| dicalcium phosphate dihydrate | 1% to 5% |
| artificial flavor | 0.1% to 1% |
| aloe vera gel | 0.1% to 0.3% |
| phosphoric acid | 0.1% to 1% |
| sucralose | 0.1% |
| mulberry root extract | 0.1% to 1% |

According to the first alternative mode, the artificial flavor is preferably the predetermined amount of Noville Extreme Peppermint Flavor which is utilized to deliver a fresh and cool feeling to the user of the present invention.

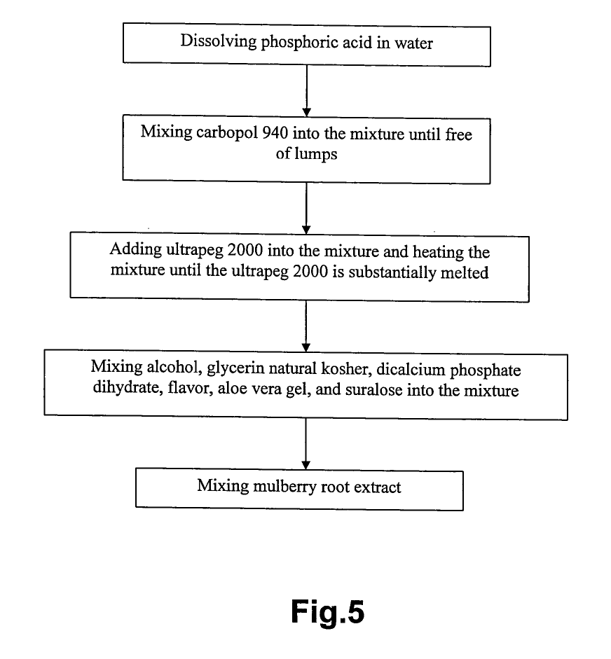

Referring to FIG. 5 of the drawings, a flow diagram outlining the manufacturing method of the whitening compound of the present invention is illustrated. The manufacturing method comprises the steps of:
(1′) Mix a predetermined amount of the phosphoric acid of predetermined concentration with a predetermined amount of water to form a phosphoric acid solution.
(2′) Mix a predetermined amount of the carbopol 940 with the phosphoric acid solution to form a mixture with free of lumps.
(3′) Heat up a predetermined amount of ultrapeg mixed in the mixture until the ultrapeg is substantially dissolved in the mixture to form an ultrapeg based mixture.
(4′) Mix a predetermined respective amount of alcohol, glycerin natural kosher, dicalcium phosphate dihydrate, artificial flavor, aloe vera gel, carbamide peroxides, and suralose into the ultrapeg based mixture until all ingredients are substantially dissolved to form the whitening agent.
(5′) Mix a predetermined amount of mulberry root extract with the whitening agent to form the tooth whitening compound.

It is worth to mention that the carbamide peroxide can be alternatively mixed with the ultrapeg based mixture after the mulberry root extract is mixed therewith in the step (5′).

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims

1. A tooth whitening compound for whitening a tooth surface, comprising:
a whitening agent comprising a poloxamer, a hydrogen peroxide, a glycerin, a dicalcium phosphate dehydrate, a phosphoric acid, and Food, Drug & Cosmetic (FD&C) Green No.3 mixing together with a predetermined mount of water to form a whitening gel for applying on said tooth surface to process a teeth whitening reaction between said whitening agent and said tooth surface; and
a whitening catalyst having a predetermined amount of amino acids mixing with said whitening agent to chemically react with said hydrogen peroxide for stabilizing said teeth whitening reaction.

2. The tooth whitening compound, as recited in claim 1, wherein 0.1 to 1% by weight of said whitening catalyst is mixed with said whitening agent.

3. The tooth whitening compound, as recited in claim 1, wherein said whitening catalyst is mulberry root extract.

4. The tooth whitening compound, as recited in claim 2, wherein said whitening catalyst is mulberry root extract.

5. The tooth whitening compound, as recited in claim 3, wherein said whitening catalyst is manufactured through a process which comprises the steps of:
(a) preparing a predetermined amount of natural mulberry roots;
(b) soaking said natural mulberry roots in water for a predetermined period of time to form a mulberry roots extract solution; and
(c) extracting said mulberry roots extract solution to form said mulberry root extract by:
(c.1) filtering said mulberry roots from said mulberry roots extract solution;
(c.2) evaporating said water from said mulberry roots extract solution to form a saturated mulberry roots solution; and
(c.3) crystallizing said saturated mulberry roots solution to form said mulberry roots extract.

6. The tooth whitening compound, as recited in claim 4, wherein said whitening catalyst is manufactured through a process which comprises the steps of:
(a) preparing a predetermined amount of natural mulberry roots;
(b) soaking said natural mulberry roots in water for a predetermined period of time to form a mulberry roots extract solution; and
(c) extracting said mulberry roots extract solution to form said mulberry root extract by:
(c.1) filtering said mulberry roots from said mulberry roots extract solution;
(c.2) evaporating said water from said mulberry roots extract solution to form a saturated mulberry roots solution; and
(c.3) crystallizing said saturated mulberry roots solution to form said mulberry roots extract.

7. The tooth whitening compound, as recited in claim 4, is manufactured through a process which comprises the steps of:
(A) mixing a predetermined amount of said phosphoric acid with said water to form a phosphoric acid solution;
(B) mixing a predetermined amount of said poloxamer with said phosphoric acid solution to form a mixture with free of lumps;
(C) mixing a predetermined amount of said glycerin, said dicalcium phosphate dehydrate, said hydrogen peroxides, and said Food, Drug & Cosmetic (FD&C) Green No.3 with said mixture to form said whitening agent; and
(D) mixing said mulberry roots extract with said whitening agent to form said tooth whitening compound.

8. The tooth whitening compound, as recited in claim 6, is manufactured through a process which comprises the steps of:
(A) mixing a predetermined amount of said phosphoric acid with said water to form a phosphoric acid solution;
(B) mixing a predetermined amount of said poloxamer with said phosphoric acid solution to form a mixture with free of lumps;
(C) mixing a predetermined amount of said glycerin, said dicalcium phosphate dehydrate, said hydrogen peroxides, and said Food, Drug & Cosmetic (FD&C) Green No.3 with said mixture to form said whitening agent; and
(D) mixing said mulberry roots extract with said whitening agent to form said tooth whitening compound.

9. The tooth whitening compound, as recited in claim 2, wherein 10 to 30% by weight of said poloxamer, 3 to 6% by weight of said hydrogen peroxide, 7 to 10% by weight of said glycerin, 1 to 2% by weight of said dicalcium phosphate dehydrate, 1 to 2% by weight of said phosphoric acid, 0.1 to 1% by weight of said Food, Drug & Cosmetic Green No. 3, and 50 to 70% by weight of said water are mixed to form said whitening agent.

10. The tooth whitening compound, as recited in claim 4, wherein 10 to 30% by weight of said poloxamer, 3 to 6% by weight of said hydrogen peroxide, 7 to 10% by weight of said glycerin, 1 to 2% by weight of said dicalcium phosphate dehydrate, 1 to 2% by weight of said phosphoric acid, 0.1 to 1% by weight of said Food, Drug & Cosmetic Green No. 3, and 50 to 70% by weight of said water are mixed to form said whitening agent.

11. The tooth whitening compound, as recited in claim 8, wherein 10 to 30% by weight of said poloxamer, 3 to 6% by weight of said hydrogen peroxide, 7 to 10% by weight of said glycerin, 1 to 2% by weight of said dicalcium phosphate dehydrate, 1 to 2% by weight of said phosphoric acid, 0.1 to 1% by weight of said Food, Drug & Cosmetic Green No. 3, and 50 to 70% by weight of said water are mixed to form said whitening agent.

12. A tooth whitening compound for whitening a tooth surface, comprising:
a whitening agent comprising an ultrapeg, a carbamide peroxide, a glycerin natural kosher, a dicalcium phosphate dihydrate, a carbopol, a phosphoric acid, and a sucralose mixing together with a predetermined mount of water to form a whitening gel for applying on said tooth surface to process a teeth whitening reaction between said whitening agent and said tooth surface; and
a whitening catalyst having a predetermined amount of amino acids mixing with said whitening agent to chemically react with said carbamide peroxide for stabilizing said teeth whitening reaction.

13. The tooth whitening compound, as recited in claim 12, wherein 0.1 to 1% by weight of said whitening catalyst is mixed with said whitening agent.

14. The tooth whitening compound, as recited in claim 12, wherein said whitening catalyst is mulberry root extract.

15. The tooth whitening compound, as recited in claim 13, wherein said whitening catalyst is mulberry root extract.

16. The tooth whitening compound, as recited in claim 14, wherein said whitening catalyst is manufactured through a process which comprises the steps of:
(a) preparing a predetermined amount of natural mulberry roots;
(b) soaking said natural mulberry roots in water for a predetermined period of time to form a mulberry roots extract solution; and
(c) extracting said mulberry roots extract solution to form said mulberry root extract by:
(c.1) filtering said mulberry roots from said mulberry roots extract solution;
(c.2) evaporating said water from said mulberry roots extract solution to form a saturated mulberry roots solution; and
(c.3) crystallizing said saturated mulberry roots solution to form said mulberry roots extract.

17. The tooth whitening compound, as recited in claim 15, wherein said whitening catalyst is manufactured through a process which comprises the steps of:
(a) preparing a predetermined amount of natural mulberry roots;
(b) soaking said natural mulberry roots in water for a predetermined period of time to form a mulberry roots extract solution; and
(c) extracting said mulberry roots extract solution to form said mulberry root extract by:
(c.1) filtering said mulberry roots from said mulberry roots extract solution;
(c.2) evaporating said water from said mulberry roots extract solution to form a saturated mulberry roots solution; and
(c.3) crystallizing said saturated mulberry roots solution to form said mulberry roots extract.

18. The tooth whitening compound, as recited in claim 15, is manufactured through a process which comprises the steps of:
(A) mixing a predetermined amount of said phosphoric acid with said water to form a phosphoric acid solution;
(B) mixing a predetermined amount of said carbopol with said phosphoric acid solution to form a mixture with free of lumps;
(C) heating up a predetermined amount of said ultrapeg mixed in said mixture until said ultrapeg is substantially dissolved in said mixture to form an ultrapeg based mixture;
(D) mixing a predetermined amount of said glycerin natural kosher, said dicalcium phosphate dehydrate, said carbamide peroxides, and said suralose with said ultrapeg based mixture to form said whitening agent; and
(E) mixing said mulberry roots extract with said whitening agent to form said tooth whitening compound.

19. The tooth whitening compound, as recited in claim 17, is manufactured through a process which comprises the steps of:
(A) mixing a predetermined amount of said phosphoric acid with said water to form a phosphoric acid solution;
(B) mixing a predetermined amount of said carbopol with said phosphoric acid solution to form a mixture with free of lumps;
(C) heating up a predetermined amount of said ultrapeg mixed in said mixture until said ultrapeg is substantially dissolved in said mixture to form an ultrapeg based mixture;
(D) mixing a predetermined amount of said glycerin natural kosher, said dicalcium phosphate dehydrate, said carbamide peroxides, and said suralose with said ultrapeg based mixture to form said whitening agent; and
(E) mixing said mulberry roots extract with said whitening agent to form said tooth whitening compound.

20. The tooth whitening compound, as recited in claim 13, wherein 20 to 30% by weight of said ultrapeg, 8 to 22% by weight of said carbamide peroxide, 10 to 20% by weight of said glycerin natural kosher, 1 to 5% by weight of said dicalcium phosphate dehydrate, 10 to 30% by weight of said carbopol, 0.1 to 1 % of said phosphoric acid, 0.1% by weight of said sucralose, and 20 to 50% by weight of said water are mixed to form said whitening agent.

21. The tooth whitening compound, as recited in claim 15, wherein 20 to 30% by weight of said ultrapeg, 8 to 22% by weight of said carbamide peroxide, 10 to 20% by weight of said glycerin natural kosher, 1 to 5% by weight of said dicalcium phosphate dehydrate, 10 to 30% by weight of said carbopol, 0.1 to 1 % of said phosphoric acid, 0.1% by weight of said sucralose, and 20 to 50% by weight of said water are mixed to form said whitening agent.

22. The tooth whitening compound, as recited in claim 19, wherein 20 to 30% by weight of said ultrapeg, 8 to 22% by weight of said carbamide peroxide, 10 to 20% by weight of said glycerin natural kosher, 1 to 5% by weight of said dicalcium phosphate dehydrate, 10 to 30% by weight of said carbopol, 0.1 to 1 % of said phosphoric acid, 0.1% by weight of said sucralose, and 20 to 50% by weight of said water are mixed to form said whitening agent.

23. A process for manufacturing a mulberry root extract as a whitening catalyst of a tooth whitening compound, comprising the steps of:
(a) preparing a predetermined amount of natural mulberry roots;
(b) soaking said natural mulberry roots in water for a predetermined period of time to form a mulberry roots extract solution; and
(c) extracting said mulberry roots extract solution to form said mulberry root extract.

24. The process, as recited in claim 23, wherein the step (c) further comprises the sub-steps of:
(c.1) filtering said mulberry roots from said mulberry roots extract solution;
(c.2) evaporating said water from said mulberry roots extract solution to form a saturated mulberry roots solution; and
(c.3) crystallizing said saturated mulberry roots solution to form said mulberry roots extract.
